(19) United States
(12) Patent Application Publication
(10) Pub. No.: US 2014/0379085 A1
(43) Pub. Date: Dec. 25, 2014

(54) REVERSE CAGE INTERVERTEBRAL FUSION IMPLANTS

(71) Applicant: Institute for Musculoskeletal Science and Education, Ltd., Radnor, PA (US)

(72) Inventors: William Duffield, Collegeville, PA (US); Joel Torretti, State College, PA (US)

(21) Appl. No.: 14/309,439

(22) Filed: Jun. 19, 2014

Related U.S. Application Data
(60) Provisional application No. 61/837,342, filed on Jun. 20, 2013.

(51) Int. Cl. A61F 2/44

(52) U.S. Cl. CPC A61F 2/447; A61F 2002/30576
USPC 623/17.16

(57) ABSTRACT

Low-profile reverse cage intervertebral implants are provided having an endplate positioned on the posterior side of the cage. Having the endplate positioned posteriorly provides several advantages including placement of fastening means away from blood vessels anterior to the intervertebral region as well as placement of bone screws to prevent backing out. The endplates are overall smaller than corresponding endplates for traditional (anterior) positioning. Implants are provided having various means for securing the implant in the intervertebral space, including one or more blades and/or one or more bone screws.

### CROSS-REFERENCE TO RELATED APPLICATIONS

This application claims priority to U.S. Provisional Application Ser. No. 61/837,342, filed Jun. 20, 2013, the disclosure of which is incorporated herein by reference in its entirety.

### FIELD OF THE INVENTION

The present disclosure is generally in the field of devices and implants for positioning and immobilizing two or more adjacent vertebra.

### BACKGROUND OF THE INVENTION

The spinal disc and/or vertebral bodies can be displaced or damaged due to trauma, disease, degenerative defects, or wear over an extended period of time. One result of this displacement or damage to a spinal disc or vertebral body can be chronic back pain. Intervertebral disc degeneration impacts the majority of people, with more than 60% of patients beyond age 40 displaying some level of disc degeneration on an MRI. This is most prevalent in the lumbar spine.

The standard treatment for chronic pain related to damaged or displaced discs is lumbar spinal fusion. There are two main types of lumbar spinal fusion, which can be used in conjunction with each other. Posterolateral fusion places the bone graft between the transverse processes in the back of the spine. These vertebrae are then fixed in place with screws and/or wire through the pedicles of each vertebra attaching to a metal rod on each side of the vertebrae. Interbody fusion places the bone graft between the vertebra in the area usually occupied by the intervertebral disc. In preparation for the spinal fusion, the inner nucleus pulposus is removed entirely. A device such as an intervertebral cage or implant can be placed between the vertebra to restore proper spine alignment and disc height.

Cervical spinal fusions can be performed on the neck. Bone, metal plates, or screws can make a bridge between adjacent vertebrae. In extreme cases, whole vertebrae can be removed before the fusion occurs. Usually, however, only the intervertebral disk is removed, and the bone or PEEK graft is subsequently inserted, allowing for the vertebrae to eventually heal together. Cervical spinal fusion can be performed for several reasons. Following injury, this surgery can help stabilize the neck and prevent fractures of the spinal column which could damage the spinal cord. It can also treat misaligned vertebrae or as a follow up for other spinal injuries. Cervical spinal fusion can remove or reduce pressure on nerve roots caused by bone fragments or ruptured intervertebral disks.

The success or failure of spinal fusion depends on several factors. For instance the spacer or cage used to fill the space left by the removed disc and bony anatomy must be sufficiently strong to support the spine under a wide range of loading conditions. The implant should also be configured to remain in place once it has been positioned in the spine by the surgeon. Additionally the bone graft materials used should be biocompatible and promote bony ingrowth.

Common causes of failure in spinal fusion include slippage of the implant, breakage of the plates, or the backing out of screws that secure the implant and/or bone fixation plate. Screws back out, typically as a result of the failure of the screws to achieve a sufficient purchase in the bone; although the stripping of the threads on the screws also causes this problem.

The implant and/or the bone fixation plate should restore as much as possible the natural curvature and range of motion to the spine.

There is a need for improved devices for spinal fusion and for improved, less invasive methods for achieving spinal fusion.

Therefore, it is an object of the invention to provide improved intervertebral fusion implants that restore as much as possible the natural curvature of the spinal region.

It is further an object of the invention to provide improved intervertebral fusion implants that remain in place following implantation.

It is further an object of the invention to provide improved and safer methods for spinal fusion, in particular for lumbar or cervical spinal fusion.

### SUMMARY OF THE INVENTION

Low-profile, reverse cage intervertebral fusion implants for spinal fusion, especially in the lumbar spine, kits containing the implants, including suitable stabilization means, and methods of using the implants and kits are described herein. The implants have a spacer anteriorly located and an endplate posteriorly located. The reverse cage implants are placed, typically via an anterior approach, although other approaches can also be employed, within the intervertebral space between adjacent superior and inferior vertebra. The implants can restore or substantially restore the natural shape and curvature locally of the vertebral region while promoting growth of bone and fusion of adjacent vertebral bodies.

Preferably the implant also contains suitable stabilization means to secure the implant to the intervertebral space. Suitable stabilization means include, but are not limited to, blades and bone screws in various orientations.

In one embodiment, the stabilization means are two blades. The blades can engage on one end the endplate of the intervertebral implant and, on an opposite end, the vertebral body of the adjacent superior or inferior vertebra. In one embodiment, each of the blades engages the endplate via a fastener slot positioned near the sinister and dexter ends of the endplate. The slots and/or blades are oriented at such angles that the blades cross the sagittal plane, such as at an angle between 0° and 75°, preferably 15° to 60°, more preferably from about 30° to about 45°, more preferably at about 35° relative to the sagittal plane. In another embodiment, the two blades are positioned symmetrically about the sagittal plane, with the first blade extending superiorly from the implant, and the second blade extending inferiorly from the implant. Typically, the blades are parallel to the sagittal plane; however, the blades may be offset from the sagittal plane by a suitable angle, such as ranging from about 15° to about 45°. In this embodiment, both of the blades extend anteriorly to engage the adjacent superior and inferior vertebral bodies, respectively. For example, the blades may be aligned at an angle relative to the transverse plane, such as from about 15° to 75°, preferably from about 30° to about 40° relative to the transverse plane.

In an alternative embodiment, the stabilization means are bone screws. The endplate may contain two or more screw holes positioned for receiving the bone screws, preferably two bone screws. The screw holes can be positioned near the sinister and dexter ends of the endplate. The screw holes are oriented such that a first bone screw can engage the adjacent superior vertebral body and a second bone screw can engage the adjacent inferior vertebral body.

In yet another embodiment, the stabilization means is a bridge and one or more bone screws. The bridge contacts both the anterior wall of the spacer portion and the endplate. The bridge may contain a screw hole positioned for receiving a bone screw and oriented such that the bone screw can engage the vertebral bodies of both the adjacent superior and inferior vertebra.

### BRIEF DESCRIPTION OF THE DRAWINGS

FIG. 1 depicts a sectional dexter view onto the sagittal plane of an idealized intervertebral space. For clarity the intervertebral implant is not drawn.

FIGS. 2A-2F depict different views of one embodiment of a low-profile reverse cage intervertebral implant having two blades positioned sinisterly and dexterly for securing the implant in the intervertebral space. FIG. 2A is a perspective view of the reverse cage intervertebral implant. FIG. 2B is a top plan view of the reverse cage intervertebral implant from FIG. 2A depicting the interior void and the relative orientation of the blades. FIG. 2C is a dexter elevation view of the reverse cage intervertebral implant from FIG. 2A. FIG. 2D is an anterior elevation view of the reverse cage intervertebral implant from FIG. 2A. FIG. 2E is a perspective view depicting placement of the implant from FIG. 2A in the intervertebral space. Only the adjacent inferior vertebra is depicted for clarity. FIG. 2F is an exploded view of the implant depicted in FIG. 2A.

FIGS. 3A-3D depict different views of one embodiment of a low-profile reverse cage intervertebral implant having two blades positioned superiorly and inferiorly for securing the implant in the intervertebral space. FIG. 3A is a perspective view of the low-profile reverse cage intervertebral implant depicting the spacer, endplate, and blades. FIG. 3B is an exploded view of the implant from FIG. 3A depicting the fastener slots for engaging the blades with the endplate. FIG. 3C is a perspective view depicting placement of the implant from FIG. 3A in the intervertebral space. Only the adjacent inferior vertebra is depicted for clarity. FIG. 3D is a dexter cross-sectional view onto the sagittal plane depicting the placement of the implant in FIGS. 3A-3C in the intervertebral space.

FIGS. 4A-4E depict different views of one embodiment of a low-profile reverse cage intervertebral implant having two bone screws for securing the implant in the intervertebral space. FIG. 4A is a perspective view of the implant showing the spacer and the endplate. The bone screws are omitted for clarity. FIG. 4B is an exploded view of the implant from FIG. 4A. FIG. 4C is a perspective view depicting placement of the implant from FIG. 4A in the intervertebral space. Only the adjacent inferior vertebra is depicted for clarity. FIG. 4D is a top plan view depicting the placement of the implant from FIG. 4A in the intervertebral space. Only the adjacent inferior vertebra is depicted for clarity. FIG. 4E is a cross-sectional view taken along lines A-A of FIG. 4D depicting the relative orientation of the bone screw engaging the adjacent superior vertebra (not pictured) for securing the implant in the intervertebral space.

FIGS. 5A-5G depict different views of one embodiment of a low-profile reverse cage intervertebral implant having a single bone screw for securing the implant in the intervertebral space. FIG. 5A is a perspective view of the implant depicting the implant having a bridge and a single central bone screw inserted therein. FIG. 5B is a dexter elevation view of the implant from FIG. 5A. FIG. 5C is a top plan view of the implant from FIG. 5A. FIG. 5D is an anterior elevation view of the implant from FIG. 5A. FIG. 5E is an anterior elevation view of a section of the spine showing placement of the implant from FIG. 5A in the intervertebral space. FIG. 5F is a cross-sectional view taken along lines A-A from FIG. 5E depicting the placement from FIG. 5E of the implant in the intervertebral space. FIG. 5G is a detailed sectional view of the region B indicated in FIG. 5F.

### DETAILED DESCRIPTION OF THE INVENTION

Low-profile reverse cage intervertebral fusion implants are disclosed herein. The reverse cage implants include a spacer region anteriorly located and an endplate posteriorly located. Reverse cage intervertebral implants are useful for spinal fusion, especially in the lumbar spine. The reverse cage implants are placed within the intervertebral space between adjacent superior and inferior vertebra. Typically an anterior approach is used for placement of the implant, however other approaches can also be employed. The implants restore or largely restore the natural shape and curvature locally of the vertebral region while promoting growth of bone and fusion of adjacent vertebral bodies.

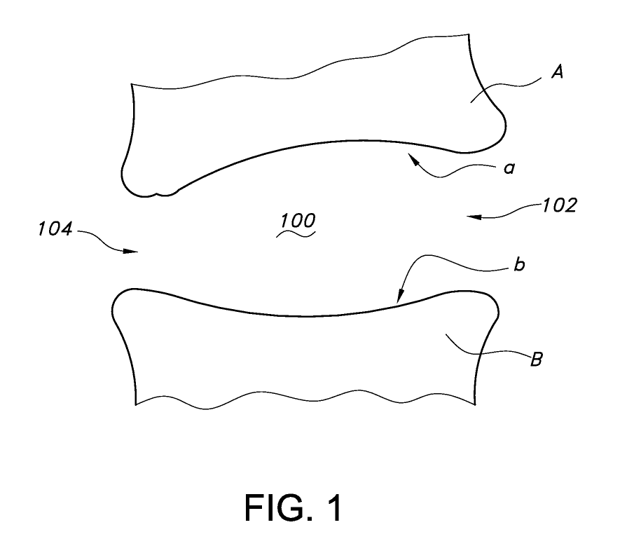

Some basic terms and measures used to characterize the regions and dimension of the intervertebral space are depicted in FIG. 1. FIG. 1 is a dexter projection into the sagittal plane of the body of the intervertebral space 100 between an idealized superior (upper) vertebra A and an idealized inferior (lower) vertebra B. The reverse cage intervertebral implant and the intervertebral disc is removed in FIG. 1 for clarity. The reverse cage intervertebral implants have a suitable shape and dimension so as to fit into the intervertebral space 100, engaging the superior vertebral surface a and the inferior vertebral surface b, and such that the spacer extends from the anterior region 102 of the intervertebral space 100 and engages the endplate in the posterior region 104 of the intervertebral space 100.

The anteromediolateral distance of the intervertebral space refers to the straight-line distance in the medial plane between the anterodexter contact point and the anterosinister contact point, being respectively the most dexter and sinister points on the intervertebral disc or implant lying in the medial plane and perpendicular to the first line segment connecting the anterior contact points.

### I. REVERSE CAGE INTERVERTEBRAL FUSION IMPLANTS

The reverse cage intervertebral fusion implant typically contains at least an endplate and a spacer. The endplate typically forms the posterior wall of the implant and the spacer is positioned anterior to the endplate and forms the remaining walls of the implant. The endplate and spacer combine to form the cage of the implant.

The walls created by the endplate and spacer define an interior region of the implant (interior region of the cage), the interior region typically being hollow although this need not necessarily be the case. The implant generally also contains one or more stabilization means that can be mechanical or non-mechanical (e.g. adhesive or friction fit).

The superior and inferior surfaces of the reverse cage intervertebral fusion implant have a suitable size and shape for mating with the superior and inferior vertebral bodies, and can be independently planar, concave, or convex. In some embodiments the superior surface, inferior surface, or both surfaces are slightly nonplanar, i.e. feature a slight curvature in a concave or convex manner. In some embodiments the inferior surface, the superior surface, or both surfaces are convex with a convexity of at least 0.01 mm, 0.1 mm, or 0.5 mm. One or both surfaces can have a convexity not over 2.0 mm, 1.0 mm, or 0.5 mm in some embodiments. In some embodiments the superior surface, inferior surface, or both surfaces have a convexity from 0.01 mm to 2 mm, from 0.1 mm to 1.0 mm, or from 0.1 mm to 0.5 mm.

a. Endplate

The endplate serves as at least part of the posterior wall, and typically all of the posterior wall of the implant. The endplate generally has a sufficient size and shape to fit in the posterior region of the intervertebral space and to substantially restore the natural curvature of the spinal region in a human patient.

The endplate contains an inner and an outer surface. The inner surface is the surface proximal to the interior region of the cage, and the outer surface is the most posterior surface of the cage. The endplate also has an upper and a lower surface. The upper and lower surfaces form at least a portion of the superior and inferior surfaces of the cage, respectively.

Each surface of the endplate can partially or entirely include a textured (i.e. not a smooth surface) or slightly irregular surface. One or more of the surfaces may contain a plurality of sharp ridges. A textured surface can include a plurality of ridges, grooves, dimples, nodules, bumps, raised portions, and/or patterns, or any combination thereof. A textured surface can have any surface roughness. In some embodiments a textured surface has a surface roughness from 1 micron to 2 mm, from 0.01 mm to 1.5 mm, from 0.1 mm to 1.5 mm, or from 0.25 mm to 1.0 mm.

In a reverse cage intervertebral implant the endplate typically defines the shortest overall height of the cage. The endplate can be any height needed to accommodate the intervertebral space. In some cases, for an improved fit to the intervertebral space and/or to best restore the natural curvature of the lumbar spine, the endplate for a lumbar intervertebral fusion implant can have a height ranging from 3 mm to 50 mm, from 5 mm to 30 mm, from 7 mm to 23 mm, or from 7 mm to 15 mm.

The endplate typically has a width that is less than the mediolateral distance of the intervertebral space. Typical widths can be range from 15 mm to 50 mm, from 20 mm to 40 mm, or preferably from 23 mm to 33 mm. The width of the endplate is less than 50 mm for a lumbar intervertebral implant, optionally less than 40 mm, and optionally from 15 mm to 35 mm. The endplate in a reverse cage intervertebral implant is typically smaller than the spacer in both height and width, thereby better accommodating the natural shape of the intervertebral space.

b. Spacer

The spacer portion forms at least the anterior wall of the cage in a reverse cage intervertebral implant. In most cases, the spacer portion forms the anterior wall and part or all of the side (sinister and dexter) walls of the cage. The spacer generally has a suitable size and dimension to fit comfortably into the anterior region of the intervertebral space and to restore as much as possible the natural curvature of the spinal region.

The spacer usually contains at least one or more inner and one or more outer surfaces, being defined analogously as the endplate with the inner surfaces being the surfaces immediately adjacent to the interior region of the cage. The spacer also typically contains both an upper and a lower surface, the upper and lower surface forming at least a portion of the superior and inferior surfaces of the cage respectively.

The width of the anterior wall of the spacer, generally adapted to accommodate the anteromediolateral distance of the intervertebral space, is less than 100 mm. In some embodiments the width of the anterior wall of the spacer is sufficient to fit the spacer within an intervertebral space having a width of less than 80 mm, a width of 20-70 mm, or a width of 30-60 mm.

i. Textured, Featured or Irregular Surface

Each surface of the spacer can partially or entirely include a featured and/or a textured or irregular surface. The features or texture on the surface(s) increase the frictional resistance between the surfaces of the implant and the adjacent vertebral bodies compared to the same surface without the features or texture, thereby increasing the stability of the implant within the patient's spine. One or more of the surfaces can include a plurality of features such as sharp ridges. A featured surface can include a plurality of deforming features such as ridges, grooves, dimples, nodules, bumps, raised portions or patterns, or any combination thereof. A textured surface can have any surface roughness. In some embodiments a textured surface has a surface roughness from 1 micron to 2 mm, from 0.01 mm to 1.5 mm, from 0.1 mm to 1.5 mm, or from 0.25 mm to 1.0 mm.

ii. Height of the Walls in the Spacer Portion

The height of one or more of the walls can be non-uniform over the entire length of the wall. The height of one or more of the walls is selected to restore the natural geometry of the intervertebral space when the cage is in place in a patient's spine. In some embodiments, the lateral walls decrease in height when going from the anterior to the posterior, i.e. the sinister and dexter lateral walls have a tallest portion at or near the anterior wall and a lowest portion at or near the endplate.

The anterior wall of the spacer can be any height that accommodates the intervertebral space. A spacer for the lumbar spine can in some embodiments substantially restore the natural shape of the vertebral region by having an anterior wall with a height of 3-30 mm, with a height of 3-25 mm, with a height of 5-25 mm, or with a height of 8-18 mm.

iii. Relative Heights of Walls in Implant

The height of the spacer can be any height needed to best accommodate the intervertebral space. The anterior wall of the spacer typically has the greatest height relative to the other three walls; and the posterior wall has the smallest height relative to the other three walls. However, this could be different for spacers with superior or inferior surfaces with high convexity.

The endplate generally determines the posterior height of the implant. The endplate for the lumbar spine typically has a height of from 3 mm to 50 mm, from 5 mm to 30 mm, from 7 mm to 23 mm, or from 7 mm to 15 mm. Because the range of posterior heights is smaller, few differently sized endplates can sometimes be used to better accommodate a variety of intervertebral spaces with different dimensions.

c. Stabilization Means

Generally, the implant contains suitable stabilization means. Suitable stabilization means secures the implant to the intervertebral space. The stabilization means can be anything capable of mechanically engaging both the implant and the adjacent vertebral bodies in a manner that stabilizes the implant in the intervertebral space.

Suitable stabilization means may be mechanical elements, such as blades or bone screws in various orientations. Alternatively, the stabilization means may be an adhesive, such as adhesive materials or adhesive surfaces on the implant and/or endplate, or friction, such as due to the fit of the particular shape of the implant in the intervertebral space (e.g., friction fit, or “lock and key”). Optionally, the implant contains combination of different stabilization means. In preferred embodiments the stabilization means are bone screws or blades, although one skilled in the art can recognize many other alternative stabilization means. These embodiments are understood to be encompassed as well.

The stabilization means generally engages, typically on one end, the endplate. In some embodiments the stabilization means does not engage the endplate, for example in some embodiments a bridge is provided that traverses the interior region of the cage and engages one or more stabilization means. One skilled in the art is aware of numerous stabilization means and methods of securing the stabilization means. These are understood to be encompassed by some of the embodiments described herein. The stabilization means can, in some embodiments, securely engage with the endplate or the bridge using a combination of one or more pins and/or one or more screws. The endplate may include one or more features for mechanically receiving or securing the stabilization means. For example, the endplate can contain one or more screw holes capable of mechanically engaging one or more bone screws. In a reverse cage intervertebral implant, the posteriorly positioned endplate allows for placement of stabilization means that engage the endplate away from blood vessels and tissue located anterior to the intervertebral space.

The stabilization means can be one or more bone screws containing at least one threaded region capable of mechanically engaging a screw hole positioned in the endplate. The bone screw can, in some cases, have more than one threaded region, for instance the bone screw can include a first threaded region capable of mechanically engaging a screw hole in the endplate and a second threaded region capable engaging the bony vertebral body. The second threaded region can have larger threads for engaging the vertebral body or a coating for improving bone growth and adhesion to the surface, thereby preventing the screw from backing out of the site of implantation.

The stabilization means can be a blade, having at least one end capable of engaging the endplate or bridge. The blades can be solid. Alternatively, they can include one or more voids therethrough to allow bone-ingrowth to interdigitate with the blade imparting additional unity between the implanted blade and the boney environment of the adjacent vertebral body. The blade can include anti-repulsion surface features, such as serrations or shark teeth, to retain the implant in place following implantation, and prevent the blade from backing out of the bone and to allow bone growth between the teeth of the serrations. The blades may include a sharp proximal end to facilitate cutting and insertion into the bony vertebral body. The blades typically include one end (the distal end) configured to engage a fastener slot positioned on the endplate and/or the bridge, that is designed to receive and secure the end of the blade. A reverse cage intervertebral implant can include any number of blades as desired or as required by the specific application, such as 1, 2, 3, 4, or more blades. Typically two blades are used.

The blades can engage on one end the endplate of the intervertebral implant and, on an opposite end, the vertebral body of the adjacent superior or inferior vertebra. In one embodiment, each of the blades engages the endplate near the sinister and dexter ends of the endplate. The blades are oriented at such angles that the blades cross the sagittal plane. In another embodiment, the blades are positioned symmetrically about the sagittal plane, such as with a first blade positioned superiorly on the endplate, and a second blade positioned inferiorly on the endplate. In this embodiment, both of the blades extend anteriorly to engage the adjacent superior and inferior vertebral bodies, respectively. For example, the blades may be aligned at an angle with respect to the sagittal plane of the spacer from 15° to 75°.

d. Materials

The low-profile reverse cage intervertebral implants provided herein, the endplates, the spacers, etc., can be made from any suitable material having the desired mechanical properties and level of biological compatibility.

The implant, the spacer portion, the bridge portion, the endplate, the bone screws, the blades, or any combination thereof can in some embodiments be made from a thermosetting polymer. Suitable thermosetting polymers include, but are not limited to, polyetherketoneketone (PEKK) and polyetheretherketone (PEEK). PEEK is particularly suitable because its modulus of elasticity closely matches that of bone. However, PEEK is also a hydrophobic material and bacteria tend to adhere easily to these types of surfaces.

In some embodiments a thermoplastic resin material, such as PEEK, is modified to increase surface hydrophobicity and/or is coated with an antibacterial agent. Biologically stable thermosetting polymers include, but are not limited to, polyethylene, polymethylmethacrylate, polyurethane, polysulfone, polyetherimide, polyimide, ultra-high molecular weight polyethylene (UHMWPE), cross-linked UHMWPE and members of the polyaryletherketone (PAEK) family, including polyetheretherketone (PEEK), carbon-reinforced PEEK, and polyetherketoneketone (PEKK).

In some cases the implant contains a substrate material, such as titanium, onto which a thermosetting polymer is coated.

The blades and/or bone screws are typically made from a metal or metal alloy, such as stainless steel or titanium.

e. Methods of Use

The low-profile reverse cage intervertebral implants are useful for intervertebral fusion of two or more adjacent vertebral bodies, especially in the lumbar spine. Optionally, the implants are implanted in the cervical spine as part of an intervertebral fusion procedure.

The implant is configured for placement within an intervertebral space between the adjacent vertebrae previously occupied by a spinal disc. Following implantation, the low-profile reverse cage intervertebral implant provides stabilization and torsional resistance to promote fusion of adjacent vertebrae of the spine.

The implants can be positioned by any approach, although in preferred embodiments the implants are positioned in the vertebral space from an anterior approach, from an anterior-lateral approach, or from a lateral approach.

### II. EXEMPLARY EMBODIMENTS

Although the invention is illustrated and described herein with reference to various specific embodiments, the invention is not intended to be limited to the details of the particular embodiments. Therefore, while various modifications may be made in the details and within the scope and range of equivalents of the claims, these are not departing from the invention. The specific embodiments described herein are to be regarded as “illustrative of” the low-profile reverse cage intervertebral implants.

a. A Reverse Cage Intervertebral Fusion Implant Having Two Blades

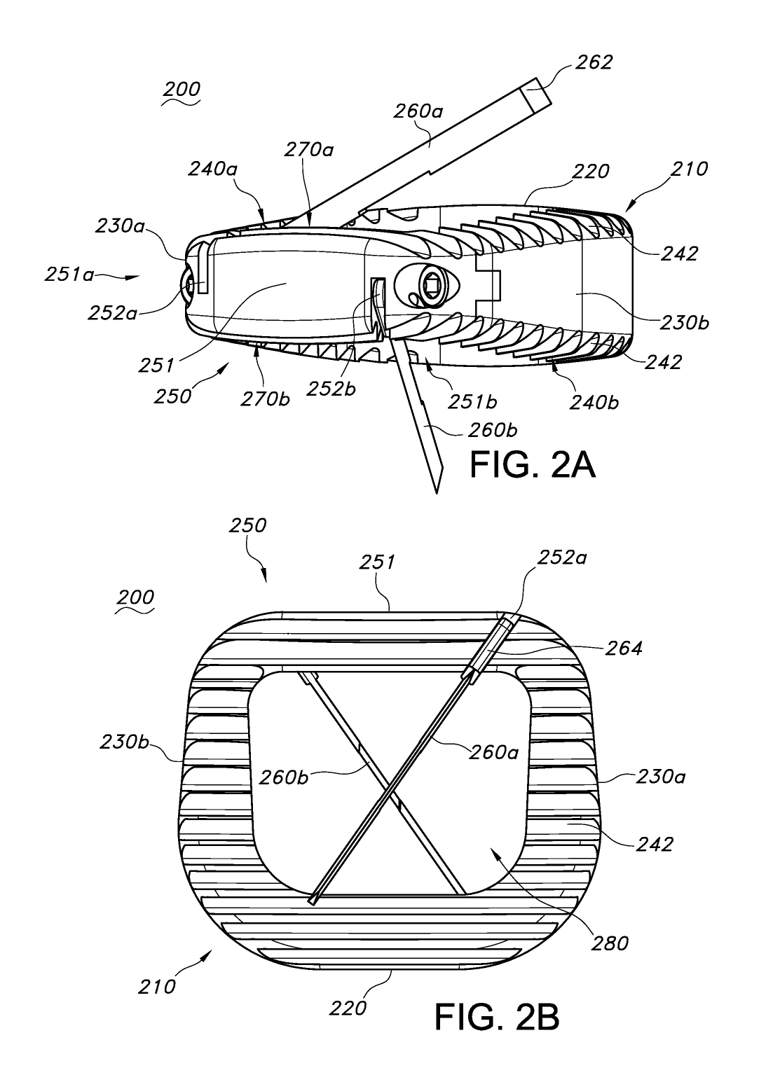

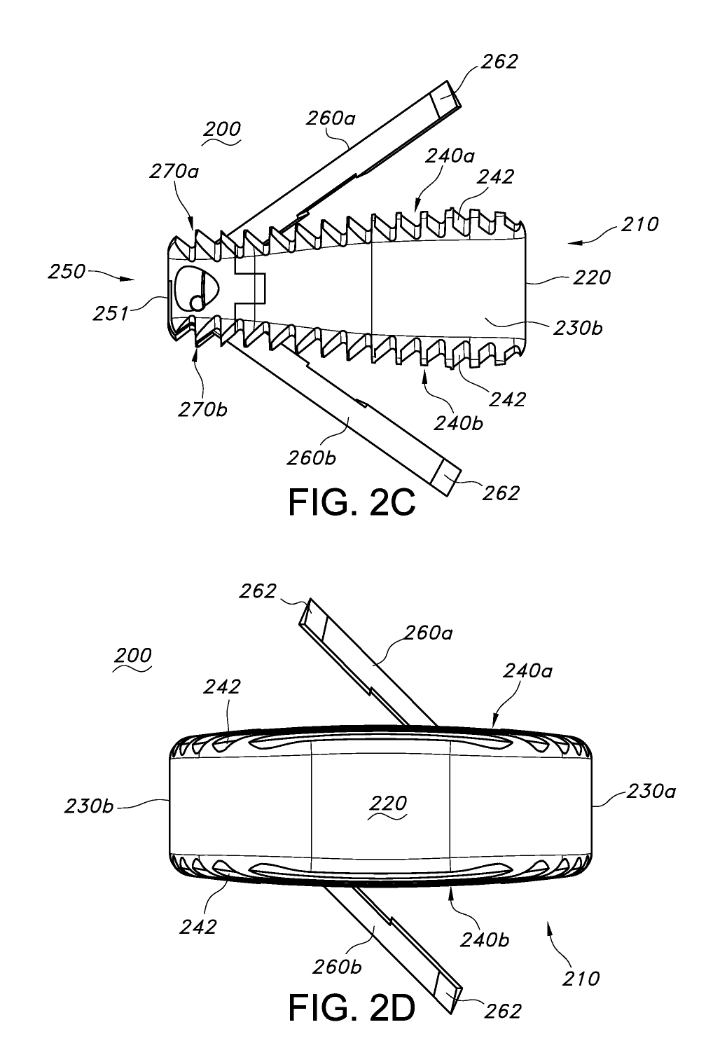

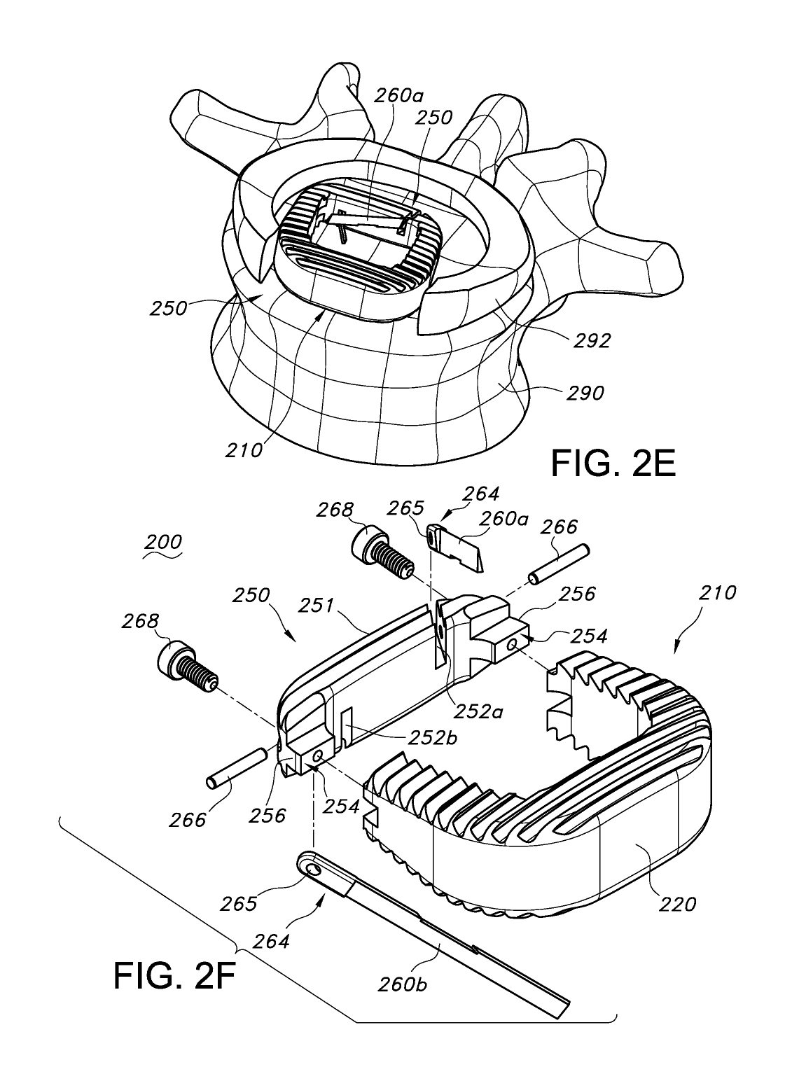

FIGS. 2A-2F depict different views of a particular embodiment of a low-profile reverse cage intervertebral implant. The implant 200 has a suitable size and shape to be positioned between two adjacent vertebrae. The implant 200 contains a spacer portion 210 and an endplate portion 250. The spacer portion, having an anterior wall 220 and a sinister lateral wall 230a and a dexter 230b lateral wall, contributes three sides of the cage. The posterior wall 251 of the cage is provided by the endplate portion 250. The walls form the boundaries of an interior void 280 that provides a space for ingrowth of the bone.

The spacer portion 210 includes a superior surface 240a and an inferior surface 240b. The inferior surface and superior surface have a suitable shape for mating with the superior and inferior vertebral bodies. The superior surface 240a and the inferior surface 240b include a plurality of sharp ridges 242. The superior surface of the endplate 270a, the inferior surface of the endplate 270b, or both surfaces can, in some embodiments, include a featured, textured, and/or irregular surface.

The lateral walls 230a and 230b decrease in height from the anterior to the posterior positions, i.e. the sinister 230a and dexter 230b lateral walls are tallest at or near the anterior wall 220 and are shortest at or near the endplate 250. The anterior wall 220 of the spacer can be any height that accommodates the intervertebral space. The spacer 210 for the lumbar spine can, in some embodiments, best restore the natural shape of the vertebral region by having an anterior wall 220 with a height of 0-30 mm, with a height of 3-25 mm, with a height of 5-25 mm, or with a height of 8-18 mm.

The anterior wall 220 has the greatest height relative to the other three walls; and the posterior wall 251 has the smallest height relative to the other three walls. The endplate 250 determines the posterior height of the implant.

The endplate 250 has a first receiving surface 254 designed to engage a second receiving surface 214 of the spacer portion 210. In this embodiment the first receiving surface 254 has one or more tongues 256 designed to engage one or more grooves 216 on the second receiving surface 214. The tongues 256 and grooves 216 can assist in securing and aligning the endplate 250 and spacer portion 210. The endplate 250 can be further secured to the spacer portion 210 by one or more small screws 268 passing through the first receiving surface 254 of the endplate 250 and the second receiving surface 214 of the spacer portion 210.

The reverse cage intervertebral implant includes one or more blades for engaging the vertebral bodies. The implant 200 includes both a superior blade 260a and an inferior blade 260b for engaging the superior (not pictured) and inferior 290 vertebral bodies respectively. The blades secure the implant against one or both of the superior and inferior 290 vertebra. The blades 260a and 260b are solid, but could include one or more voids therethrough to allow bone-ingrowth to interdigitate with the blade imparting additional unity between the implanted blade and the boney environment of the adjacent vertebral body. The blades have a sharp distal end 262, and/or a sharp proximal end 264 to facilitate cutting and insertion into the bony vertebral body.

The proximal end of the blade 264 is engaged with the endplate 250. The endplate 250 has sinister 252a and dexter 252b fastener slots. The endplate can have any number of fastener slots, as required by the application and the specific number of blades used for engaging the vertebral bodies. As depicted in FIGS. 2A-2E, the endplate 250 has two fastener slots 252a and 252b positioned on the sinister 251a and dexter 251b ends of the endplate. The fastener slots 252a and 252b allow the blades 260a and 260b to pass through the endplate and into the bony vertebral body, engaging the vertebral body and securing the intervertebral cage in place. The fastener slots 252a and 252b contain fastening means for engaging the proximal ends 264 of the blades, securing the blades with respect to the endplate 250. In this embodiment, each proximal end 264 contains a securing hole 265 for receiving a securing pin 266.

The sinister fastener slot 252a is angled such that, when secured in the sinister fastener slot 252a, the superior blade 260a progresses upward in an anterodexter direction into the vertebral body of the adjacent superior vertebra. The dexter fastener slot 252b is angled such that, when secured in the dexter fastener slot 252b, the inferior blade 260b progresses downward in an anterosinister direction into the vertebral body of the adjacent inferior vertebra 290. The blades 260a and 260b are oriented at an angle of 45° with respect to the sagittal plane, forming a crossing pattern, as depicted in FIG. 2B. However, the angle at which the blades progress from the endplate into the vertebral body can be any angle that allows the blades to engage the adjacent vertebral body.

The slots and/or blades are oriented at such angles that the blades intersect the sagittal plane. For example, the blades may be positioned at an angle between 0° and 75°, preferably 15° to 60°, more preferably from about 30° to about 45°, more preferably at about 35° relative to the sagittal plane.

As shown in FIG. 2B, when viewed from above, the angle between the blades is approximately 90°. However, the angle between the blades can range from 15° to 100°.

FIG. 2E depicts placement of the implant 200 in the intervertebral space between adjacent superior (not pictured) and inferior 290 vertebra. The implant 200 sits partially contained within the remaining annulus fibrosus 292.

b. An Alternative Reverse Cage Intervertebral Fusion Implant Having Two Blades

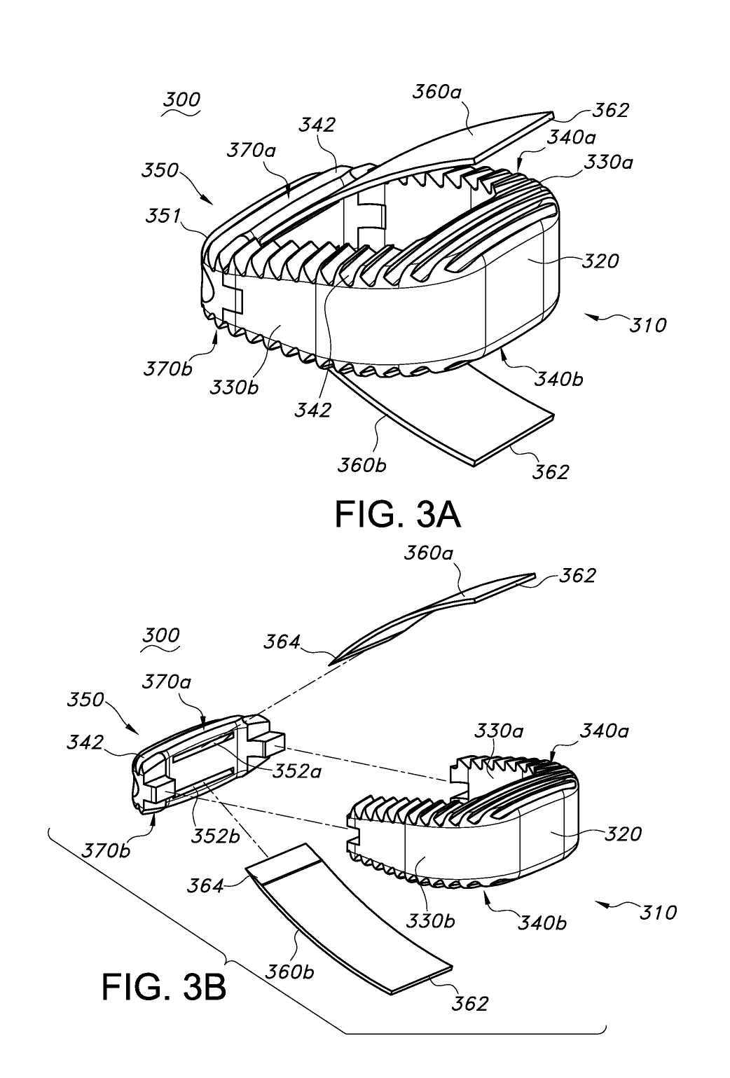

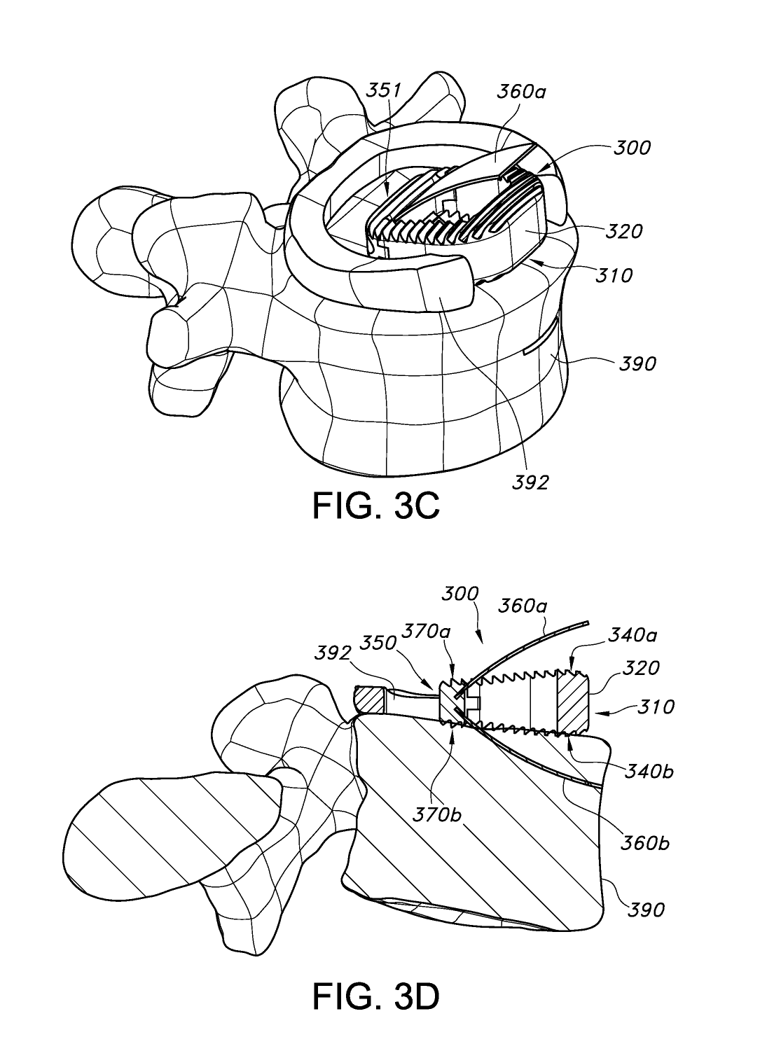

FIGS. 3A-3D depict different views of an alternative embodiment of a low-profile reverse cage intervertebral implant. The implant 300 has a sufficient size and shape to be positioned between two adjacent vertebrae. The implant 300 is primarily composed of a spacer portion 310 and an endplate portion 350. The spacer portion 310, having an anterior wall 320 and a sinister 330a and a dexter 330b lateral wall, contributes three sides of the cage. The posterior wall 351 of the cage is provided by an endplate 350. The walls form the boundaries of an interior void that promotes ingrowth of the bone.

The spacer portion 310 has both a superior surface 340a and an inferior surface 340b, which are generally planar, but can be slightly convex. The superior surface 340a and inferior surface 340b include a plurality of sharp ridges 342.

The lateral walls 330a and 330b decrease in height from the anterior to the posterior positions, i.e. the tallest portion of the sinister 330a and dexter 330b lateral walls is at or near the anterior wall 320 and the lowest portion is at or near the endplate 350. The anterior wall 320 of the spacer can be any height that accommodates the intervertebral space. The spacer 310 for the lumbar spine can, in some embodiments, best restore the natural shape of the vertebral region by having an anterior wall 320 with a height between 0 and 30 mm, or ranging from 3 to 25 mm, or ranging from 5 to 25 mm, or ranging from 8 to 18 mm.

The anterior wall 320 has the greatest height relative to the other three walls; and the posterior wall 351 has the smallest height relative to the other three walls. The endplate 350 determines the posterior height of the implant.

The implant 300 is secured by a superior blade 360a and/or an inferior blade 360b that engage the superior and inferior vertebral bodies respectively. The blades secure the implant in the superior and inferior vertebra. The blades can, in some embodiments, have a sharp distal end 362 to facilitate cutting and insertion into the bony vertebral body. The blades can be any size as needed, being long enough to sufficiently engage the adjacent vertebral body while being sufficiently short to be the least invasive.

The proximal end of the blade 364 is configured to engage the endplate 350. The endplate 350 has a superior fastener slot 352a and an inferior fastener slot 352b. In the depicted embodiment, an endplate 350 is provided having a superior fastener slot 352a capable of engaging the proximal end 364 of a superior blade 360a and an inferior fastener slot 352b capable of engaging the proximal end 364 of an inferior blade 360b. The superior fastener slot 352a is angled such that, when secured in the superior fastener slot 352a, the superior blade 360a progresses upward and in an anterior direction into the vertebral body of the adjacent superior vertebra. The inferior fastener slot 352b is angled such that, when secured in the inferior fastener slot 352b, the inferior blade 360b progresses downward and in an anterior direction into the vertebral body of the adjacent inferior vertebra. The blades 360a and 360b can be oriented parallel to the sagittal plane. Alternatively, the blades can be oriented at an angle offset from the sagittal plane, such as ranging from greater than 0° to 45° relative to the sagittal plane. The blades may be located at any angle that allows them to engage the adjacent vertebral body. For example, the blades can be aligned at an angle ranging from 15° to 45° with respect to the transverse plane of the spacer.

c. A Reverse Cage Intervertebral Fusion Implant Having Two Screws

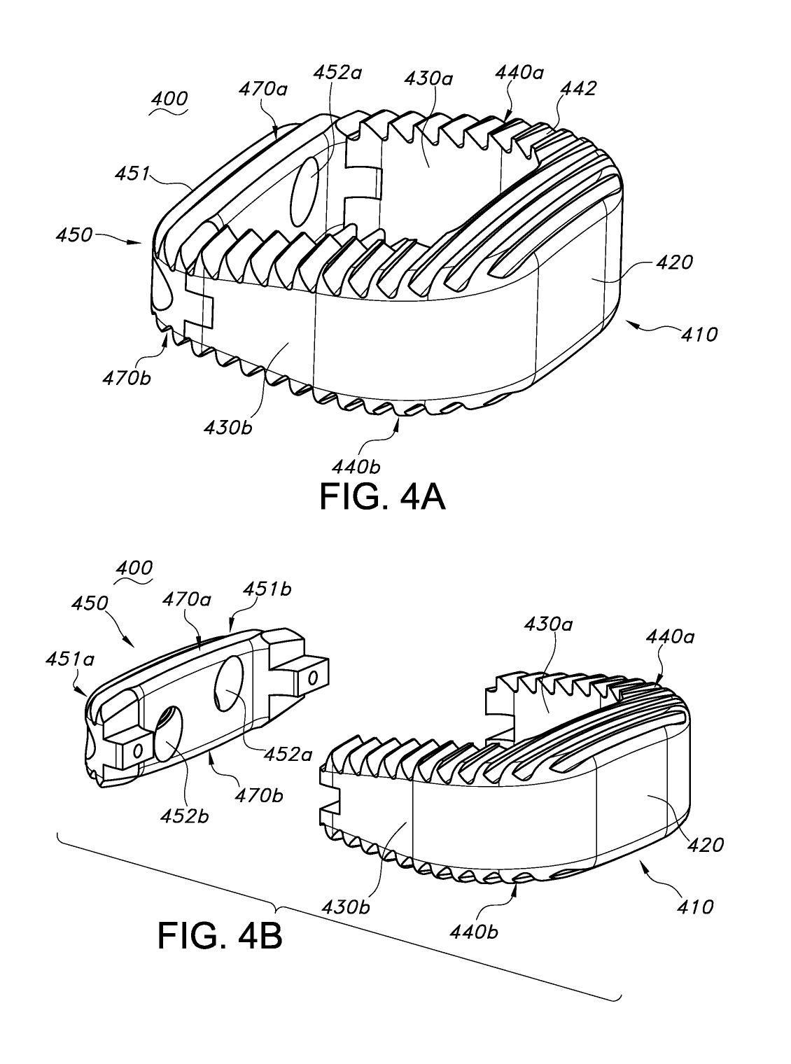

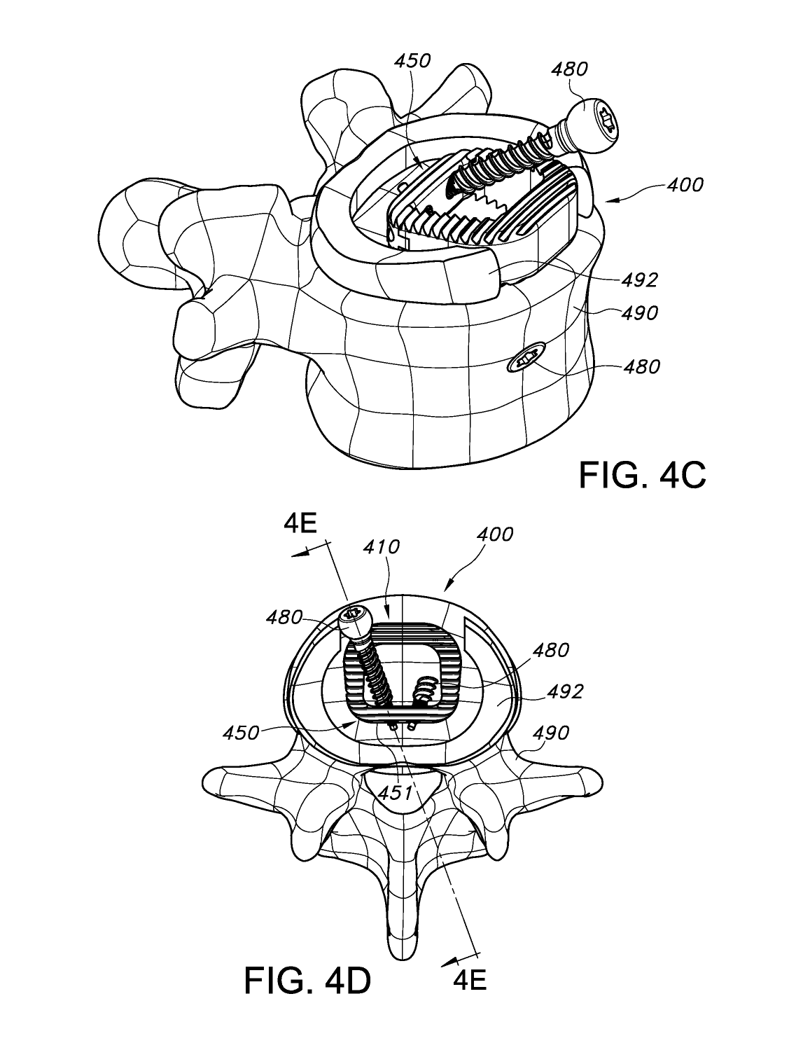

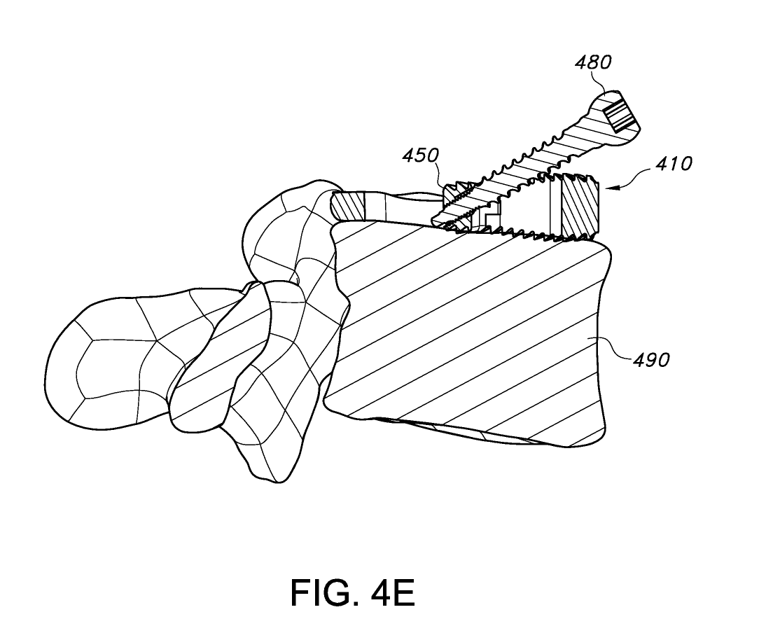

FIGS. 4A-4E depict different views of an alternative embodiment of a low-profile reverse cage intervertebral implant. The implant 400 has a suitable size to fit between two adjacent vertebrae. The implant 400 is primarily composed of a spacer portion 410 and a plate portion 450. The spacer portion, having an anterior wall 420 and a sinister 430a and a dexter 430b lateral wall, contributes three sides of the cage. The posterior wall 451 of the cage is provided by an endplate 450. The walls form the boundaries of an interior void 480 that promotes ingrowth of the bone. The spacer portion 410 has both a superior surface 440a and an inferior surface 440b are generally planar, but can be slightly convex. The superior surface 440a and inferior surface 440b include a plurality of sharp ridges 442.

The lateral walls 430a and 430b decrease in height when going from the anterior to the posterior, i.e. the sinister 430a and dexter 430b lateral walls have a tallest portion at or near the anterior wall 420 and a lowest portion at or near the endplate 450. The anterior wall 420 of the spacer can be any height that accommodates the intervertebral space. The spacer 410 for the lumbar spine can in some embodiments best restore the natural shape of the vertebral region by having an anterior wall 420 with a height between 0 and 30 mm, such as ranging from 3 to 25 mm, from 5 to 25 mm, or from 8 to 18 mm.

The anterior wall 420 has the greatest height relative to the other three walls; and the posterior wall 451 has the smallest height relative to the other three walls. The endplate 450 determines the posterior height of the implant. In the embodiment shown, the lateral walls 430a and 430b decrease in height when going from the anterior to the posterior, i.e. the sinister 430a and dexter 430b lateral walls have a tallest portion at or near the anterior wall 420 and a lowest portion at or near the endplate 450. The anterior wall 420 of the spacer can be any height that accommodates the intervertebral space. The anterior wall 420 can have a height ranging from 3 mm to 50 mm, from 5 mm to 30 mm, or from 7 mm to 23 mm.

In some embodiments the reverse cage intervertebral implant 400 is secured by one or more bone screws 480. The bone screws can, in some embodiments, be inserted from the posterior side of the implant, through the endplate, and into the bony vertebral body. In other embodiments the bone screws 480 can be inserted from the anterior side of the vertebra through the bony vertebral body and engage the endplate 450 positioned on the posterior side of the intervertebral implant.

The endplate 450 has a sinister fastener hole 452a and a dexter fastener hole 452b extending through the posterior wall of the endplate 450 and positioned on the sinister 451a and dexter 451b ends of the endplate respectively. The sinister fastener hole 452a has a longitudinal axis that extends toward the adjacent superior vertebra in the implanted state. The longitudinal axis of the dexter fastener hole 452b extends toward the adjacent inferior vertebra in the implanted state.

FIGS. 4C-4E depict placement of the implant 400 in the intervertebral space between adjacent superior (not pictured) and inferior 490 vertebra. As depicted best FIGS. 4D and 4E, a first bone screw 480 passes through the vertebral body of the adjacent superior vertebra and engages the sinister fastener hole 452a of the endplate 450. A second bone screw 480 passes through the vertebral body of the adjacent inferior vertebra 490 and engages the dexter fastener hole 452b of the endplate 450. See FIG. 4C.

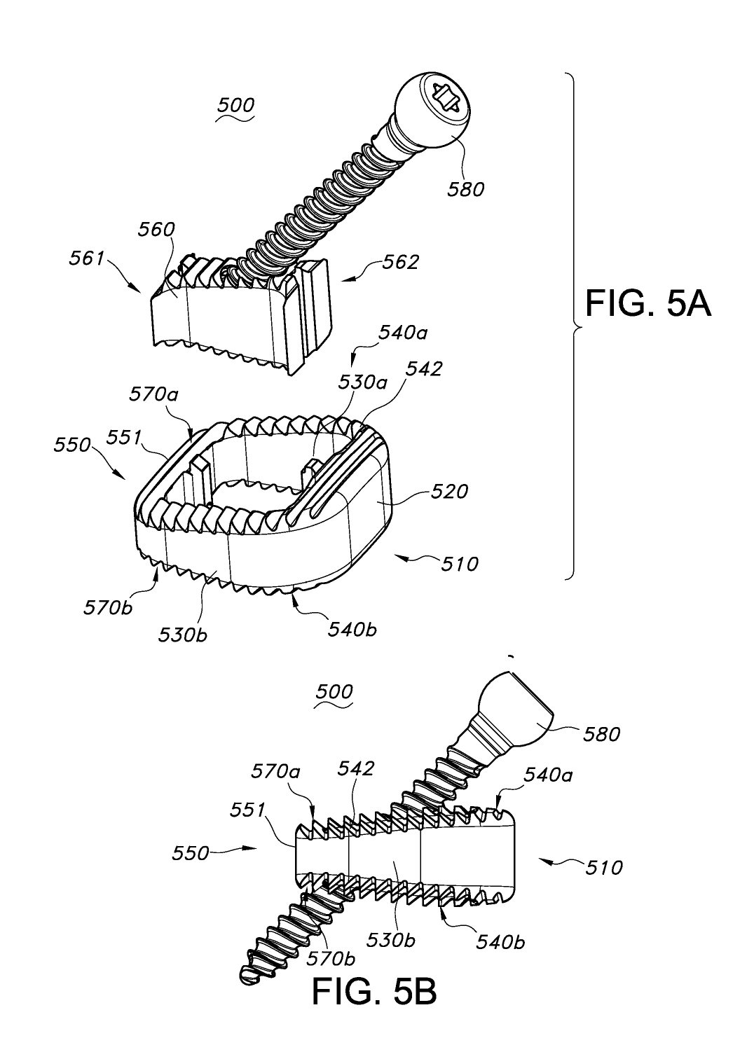

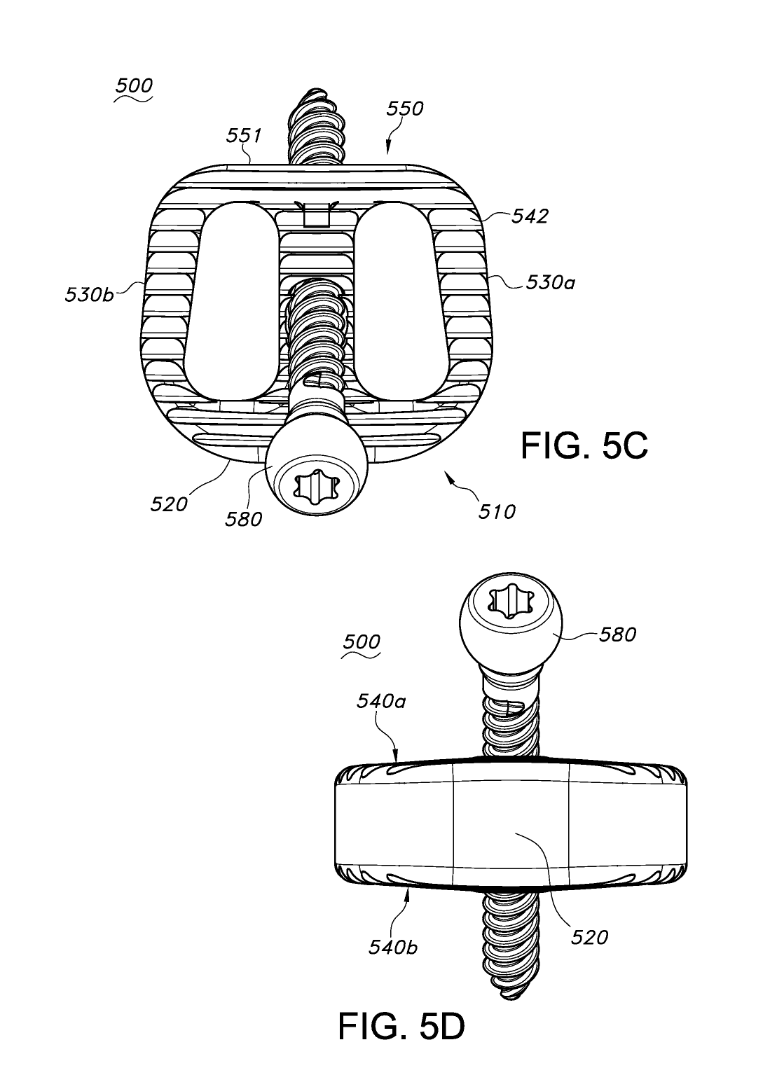

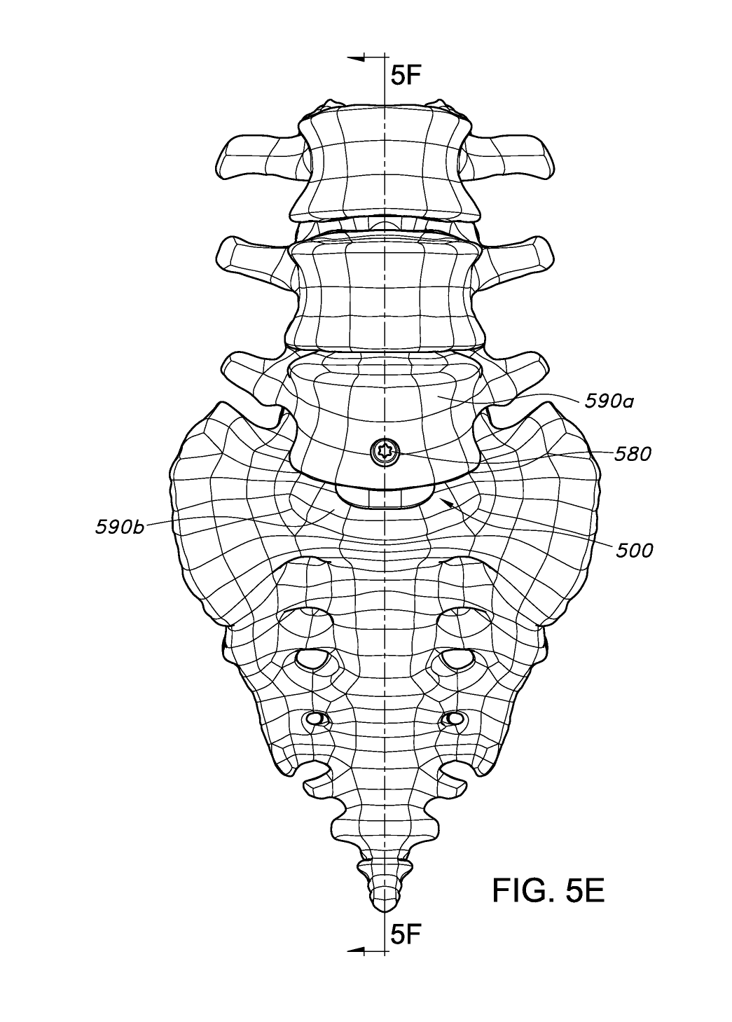

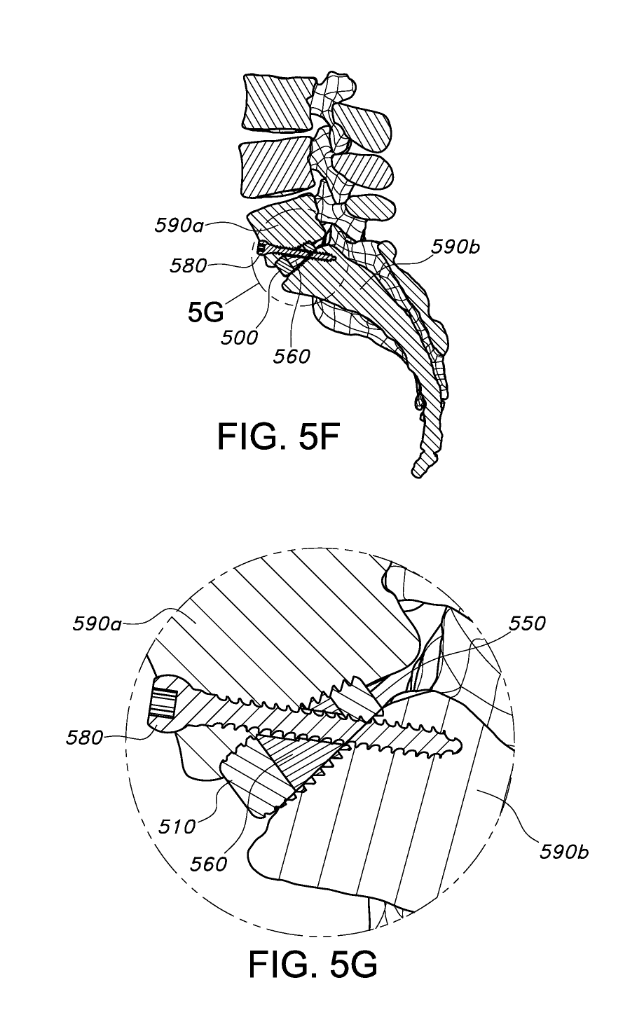

d. A Reverse Cage Intervertebral Fusion Implant Having One Screw FIGS. 5A-5G depict several views of one embodiment of a low-profile reverse cage intervertebral implant. The implant 500 is sized to fit between adjacent superior 590a and inferior 590b vertebra. The implant 500 is primarily composed of a spacer portion 510, a plate portion 550, and a bridge portion 560. The spacer portion 510, having an anterior wall 520 and sinister 530a and dexter 530b lateral walls, contributes three sides of the cage. The posterior wall 551 of the cage is provided by an endplate 550. The bridge portion 560 adjoins the endplate 550 with the anterior wall 520 of the spacer portion 510 and passes through the internal void region 580 the boundaries of which are defined by the endplate 550, and the anterior 520, sinister 530a, and dexter 530b walls of the spacer portion.

The spacer portion 510 has both a superior surface 540a and an inferior surface 540b generally planar. The superior surface of the spacer portion 540a, the inferior surface of the spacer portion 540b, the superior surface of the endplate 570a, and the inferior surface of the endplate 570b include a plurality of sharp ridges 542.

In the depicted embodiment, the lateral walls 530a and 530b decrease in height when going from the anterior to the posterior, i.e. the sinister 530a and dexter 530b lateral walls have a tallest portion at or near the anterior wall 520 and a lowest portion at or near the endplate 550. The anterior wall 520 of the spacer can be any height that accommodates the intervertebral space.

The anterior wall 520 has the greatest height relative to the other three walls; and the posterior wall 551 has the smallest height relative to the other three walls. The endplate 550 determines the posterior height of the implant. In the embodiment shown, the lateral walls 530a and 530b decrease in height when going from the anterior to the posterior, i.e., the sinister 530a and dexter 530b lateral walls have a tallest portion at or near the anterior wall 520 and a lowest portion at or near the endplate 550. The anterior wall 520 of the spacer can be any height that accommodates the intervertebral space. The anterior wall 520 can have a height ranging from 3 mm to 50 mm, from 5 mm to 30 mm, or from 7 mm to 23 mm.

The bridge portion 560 extends along the sagittal plane contacting on one end 561 the endplate 550 and on the opposite end 562 contacting the anterior wall 520 of the spacer portion 510. In other embodiments a spacer portion can be positioned out of the sagittal plane, for instance a spacer portion can be perpendicular to the sagittal plane contacting on one end the sinister wall and on the opposite end the dexter wall of a spacer portion. Therefore, in some embodiments the bridge portion need not contact the endplate. In the depicted embodiment, the bridge portion 560 contains a fastener hole for receiving a bone screw 580. The fastener hole is oriented such that the bone screw 580 passes through the bridge portion 560 and engages both the adjacent superior vertebral body 590a and the adjacent inferior vertebral body 590b.

As depicted in FIGS. 5A-5G, the screw is oriented parallel with the sagittal plane of the spacer. Alternatively, the angle of the screw is offset relative to the sagittal plane. By offsetting the angle of the screw, a surgeon can more easily avoid vessels, such as the vena cava, during implantation of the screw. Suitable angles for the screw relative to the sagittal plan range from 0° to about 45°. The screw may be oriented in a suitable angle relative to the transverse plane that allows it to engage the adjacent vertebral bodies, such as, for example, at an angle of about 45° relative to the transverse plane.

### III. THE STRUCTURE OF INTERVERTEBRAL DISCS AND IMPLANTS

There are 24 intervertebral discs in the human spine, interspersed between the vertebral bodies. The intervertebral discs can be identified by the two adjacent vertebrae, so the C6-C7 intervertebral disc lies between the two most inferior of the cervical vertebrae whereas the T12-L1 intervertebral disc lies between the inferior thoracic vertebra and the superior lumbar vertebra. The intervertebral discs generally increase in size moving down the spine, to approximately 45 mm antero-posteriorly, 64 mm laterally and 11 mm in height in the lumbar region.

The majority of disc herniation occurs in the lumbar spine, typically (˜95%) in L4-L5 or L5-S1. The cervical spine is the second most common site of spinal disc herniation, typically at C5-C6 or C6-C7. Thoracic disc herniation is the least common, occurring in less than 4% of cases.

The lumbar vertebrae graduate in size from L1 through L5. The mediolateral distance in the lumbar spine ranges from roughly 30-70 mm, with average values around 50 mm. The anteroposterior distance ranges from approximately 20-55 mm, with typical values around 35 mm.

The wedge angles (i.e., the angle between the superior and inferior surface of the intervertebral disc) typically graduate moving down the lumbar spine, increasing from 4°-10° as typical values for L1-L2 intervertebral discs to 12°-16° as typical values for L5-S1 intervertebral discs. The average wedge angle in the lumbar spine increases with age, the average across all levels of the lumbar discs being less than 10° below age 30 and increasing to over 15° beyond age 50. The average wedge angle observed from MRI and X-ray of the intervertebral space of 73 patients for T12-L1 is roughly 4°-5°, for L1-L2 is 5°-6°, for L2-L3 is 5.5°-6.5°, for L3-L4 is 6°-7°, for L4-L5 is 8°-10°, and for L5-S1 is 12°-14°. See Mark Eijkelkamp. On the Development of an Artificial Intervertebral Disc Diss., The University of Groningen, Groningen, Netherlands, 2002 and the references cited therein.

Sometimes intervertebral heights (height between the superior vertebral surface a and the inferior vertebral surface b) are reported as a single value that can be the medial height or that can be an average of the anterior and posterior height as will be apparent by context. One or more heights can also be reported as a range of values, such as a range of values observed for the different intervertebral spaces within a patient or as a range of values observed for a particular intervertebral space observed across a range of patients.

The height in the anterior region 102 for T12-L1 was observed to be approximately 8-10 mm (average 9 mm), for L1-L2 approximately 9-12 mm (average 10.5 mm), for L2-L3 approximately 10-15 mm (average 12 mm), for L3-L4 approximately 10-16 mm (average 13 mm), for L4-L5 approximately 12-16 mm (average 14 mm), and for L5-S1 approximately 9-16 mm (average 13.5 mm). The medial heights range typically from 8-10 mm (average 9 mm) for T12-L1, 10-12 mm (average 11 mm) for L1-L2, from 11-16 mm (average 13 mm) for L2-L3, from 11-17 mm (average 14) for L3-L4, from 12-16 mm (average 13 mm) for L4-L5, and from 9-13 mm (average 11 mm) for L5-S1. There is less variation in the posterior heights. The heights in the posterior region 104 observed in the same population ranged from 5-8 mm (average 6.5 mm) for T12-L1, from 6-9 mm (average 7.5 mm) for L1-L2, from 7-12 mm (average 9 mm) for L2-L3, from 7-13 mm (average 10 mm) for L3-L4, from 7-11 mm (average 9 mm) for L4-L5, from 5-9 mm (average 7 mm) for L5-S1. See Mark Eijkelkamp. On the Development of an Artificial Intervertebral Disc Diss., The University of Groningen, Groningen, Netherlands, 2002 and the references cited therein.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. An intervertebral implant for implantation in an intervertebral space between adjacent superior and inferior vertebrae, the implant comprising
a spacer portion comprising an anterior, sinister, and dexter wall; and
an endplate coupled to the spacer portion;
wherein the endplate is positioned on the posterior end of the implant.

2. The implant of claim 1, wherein each of the spacer portion and the endplate, further comprises a superior surface and an inferior surface.

3. The implant of claim 2, wherein one or more of the surfaces comprises a featured and/or a textured surface that increases the frictional resistance between the surfaces of the implant and the adjacent vertebrae compared to the same surface without the features or texture.

4. The implant of claim 2, wherein the featured surface comprises one or more features selected from the group consisting of ridges, grooves, dimples, nodules, bumps, raised portions, and patterns.

5. The implant of claim 1 further comprising one or more blades for securing the implant in the intervertebral space.

6. The implant of claim 5, wherein the implant comprises two blades, each having a proximal end and a distal end,
wherein the endplate further comprises two fastener slots that engage the proximal ends of the blades,
wherein the distal end of a first blade is configured to engage the vertebral body of the adjacent superior vertebra, and
wherein the distal end of a second blade is configured to engage the vertebral body of the adjacent inferior vertebra.

7. The implant of claim 6, wherein the fastener slots are located in the sinister and dexter ends of the endplate, and
wherein the blades are aligned at an angle with respect to the sagittal plane of the spacer from 15° to 75°.

8. The implant of claim 6, wherein the fastener slots are positioned superiorly and inferiorly along the sagittal plane of the endplate.

9. The implant of claim 1, further comprising one or more screw holes positioned to receive one or more bone screws for securing the implant in the intervertebral space.

10. The implant of claim 9, wherein the endplate comprises two screw holes.

11. The implant of claim 10, wherein the endplate comprises a first screw hole positioned to receive a first bone screw configured to engage the vertebral body of the adjacent superior vertebra, and
wherein the endplate comprises a second screw hole positioned to receive a second bone screw configured to engage the vertebral body of the adjacent inferior vertebra.

12. The implant of claim 9, further comprising a bridge portion having a first end and a second end,
wherein the bridge portion comprises one or more screw holes.

13. The implant of claim 12, wherein the first end is coupled to the endplate and the second end is coupled to the anterior wall, or
wherein the first end is coupled to the sinister wall and the second end is coupled to the dexter wall.

14. The implant of claim 13, wherein the bridge portion comprises one screw hole positioned to receive a first bone screw configured to engage both the vertebral body of the adjacent superior vertebra and the vertebral body of the adjacent inferior vertebra.

15. The implant of claim 2, wherein the superior surface of the spacer, the inferior surface of the spacer, or both surfaces comprise a convex surface.

16. The implant of claim 15, wherein the convex surface has a convexity from about 0.01 mm to 1.0 mm.

17. The implant of claim 1, wherein the endplate has a height from 7 mm to 23 mm.

18. An endplate for forming the posterior wall of an intervertebral implant for implantation in an intervertebral space between adjacent superior and inferior vertebrae, the endplate having a height from about 7 mm to about 23 mm.

19. The endplate of claim 18, wherein the endplate has a width from about 23 mm to about 33 mm.

20. The endplate of claim 18, further comprising one or more screw holes positioned to receive one or more bone screws for securing the implant in the intervertebral space.

21. The endplate of claim 18, further comprising one or more fastener slots positioned to receive one or more blades for securing the implant in the intervertebral space.

22. A kit comprising one or more spacer portions and one or more endplates,
wherein each endplate is configured to attach to the posterior end of the spacer portion and has a height ranging from about 7 mm to about 23 mm, and one or more spacers, and
wherein each spacer portion comprises an anterior, sinister, and dexter wall.

23. The kit of claim 22, comprising more than one endplate, wherein the endplates have different dimensions.
